(19) United States
(12) Patent Application Publication
(10) Pub. No.: US 2026/0007524 A1
(43) Pub. Date: Jan. 8, 2026

(54) SHOULDER STEM WITH MODULAR SENSOR

(71) Applicant: Zimmer, Inc., Warsaw, IN (US)

(72) Inventors: Kevin Miller, Wakarusa, IN (US); Kyle A. Bohnenkamper, Warsaw, IN (US); Kristoff Goson, Independence, OH (US); Raymond C. Parisi, Wakarusa, IN (US)

(21) Appl. No.: 18/841,622

(22) PCT Filed: Feb. 24, 2023
(86) PCT No.: PCT/US2023/013806

§ 371 Date: Aug. 26, 2024

Related U.S. Application Data
(60) Provisional application No. 63/319,552, filed on Mar. 14, 2022.
(60) Provisional application No. 63/409,388, filed on Sep. 23, 2022.

(51) Int. Cl. A61F 2/40; A61B 5/00; A61F 2/30; A61F 2/46

(52) U.S. Cl. CPC A61F 2/4059; A61B 5/4528; A61B 5/4851; A61B 5/6878; A61F 2/4014; A61F 2/4637; A61F 2002/30214; A61F 2002/30235; A61F 2002/30405

(57) ABSTRACT

An implant configured to be implanted into a human body including a stem (102) having an elongated body (114) with a distal portion (110). The stem includes a cavity (116) extending through the distal portion and into the elongated body. The stem also includes an attachment mechanism (128) on the opposing end (112) of the stem from the cavity. The attachment mechanism is configured to receive a second implant component (106).

### CLAIM OF PRIORITY

This patent application claims the benefit of priority to U.S. Patent Application Ser. No. 63/319,552 filed on Mar. 14, 2022, and to U.S. Patent Application Ser. No. 63/409,388 filed on Sep. 23, 2022, which are both hereby incorporated by reference herein in their entirety.

### BACKGROUND

The shoulder joint is a complex joint with the scapula, clavicle, and humerus all coming together to enable a wide range of movement, at least in a properly functioning joint. In a properly functioning shoulder joint, the head of the humerus fits into a shallow socket in the scapula, typically referred to as the glenoid. Articulation of the shoulder joint involves movement of the humeral head in the glenoid, with the structure of the mating surfaces and surrounding tissues providing a wide range of motion.

The shoulder joint can undergo degenerative changes caused by various issues, such as rheumatoid arthritis, osteoarthritis, rotator cuff arthroplasty, vascular necrosis, or bone fracture. When severe joint damage occurs, and no other means of treatment is found to be effective, a total, partial, or reverse shoulder replacement or reconstruction may be necessary. Total shoulder replacements can involve a humeral prosthetic, including a stem and a head portion used to replace the natural humeral head. Total shoulder replacements will also typically include resurfacing of the glenoid with a prosthetic implant. The glenoid implant will generally include an articulating cup shaped to receive the prosthetic humeral head. A reverse shoulder replacement (arthroplasty) involves a different set of humeral and glenoid replacement prosthetics. In a reverse shoulder replacement, the humeral component includes a cup-shaped articular surface attached to a stem implanted into the humerus, while a spherical glenoid component is used to provide an articular surface for the humeral cup.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a schematic diagram of a smart implant installed within a human body.

FIG. 2 illustrates a schematic diagram of a plurality of stems.

FIG. 3 illustrates a perspective view of a sensor assembly.

FIG. 4 illustrates a schematic diagram of a cross-sectional view of a sensor assembly installed in an implant stem.

FIG. 5 illustrates a schematic diagram of a first sensor assembly.

FIG. 6 illustrates a schematic diagram of a second sensor assembly.

FIG. 7 illustrates a schematic diagram of a system including a first plurality of stems and a second portion of the plurality of stems.

FIG. 8 illustrates a schematic diagram of an implant and a sizing fixture.

FIG. 9 illustrates a perspective view of an implant plug.

FIG. 10 illustrates a cross-sectional view of an implant plug installed in an implant stem.

FIG. 11 illustrates another example of an implant plug.

FIG. 12 illustrates another example of a sensor assembly.

FIG. 13 illustrates another example of one of a plurality of stems.

FIG. 14 illustrates a cross-sectional view of the sensor assembly from FIG. 12 installed within the plurality of stems from FIG. 13.

FIG. 15 illustrates another example of a plurality of stem with a sensor assembly installed therein.

FIG. 16 illustrates another example of a sensor assembly during installation in another example of a plurality of stems.

FIG. 17 illustrates the sensor assembly from FIG. 16 installed in the plurality of stems from FIG. 16.

### DETAILED DESCRIPTION

This disclosure relates generally to a system configured to be implanted into a human body (hereinafter referred to as “system”). The system can include a plurality of implant stems (hereinafter referred to as “implant stems”). The implant stems can be different sizes (e.g., a different diameter around the implant stem). The system can include multiple implant stems with each of the implant stems being a different size from the other implant stems. Although being described in reference to a system for a shoulder arthroplasty, the apparatuses and systems can be used for implants in other joints such as stems for the knee, hip, or the like.

The system can also include a sensor assembly that can be inserted into any one of the implant stems. The sensor assembly can include a plurality of different types of sensors (e.g., an accelerometer, a gyro sensor, a piezoelectric sensor, or any combination thereof, including any other sensor that can be used to detect motion within a body) that can log, and store information. The sensor assembly can also include an antenna that can communicate the stored information to an external controller. The sensor assembly can include a nose cone that includes a diameter. In another example, the system can include multiple sensor assemblies. Each sensor assembly of the multiple sensor assemblies can have a nose cone with a different diameter.

The system can also include at least one implant plug (hereinafter referred to as “implant plug”) that can be installed into any one of the implant stems. Each sensor and each implant plug can include a mechanical interface that allows each sensor and each implant plug to be installed and removed from any of the implant stems using a tool that includes a mechanical interface that is complementary to the mechanical interface on each sensor and each implant plug. When inserted in the implant stem, the implant plug can define a shape of an implant plug without a cavity for a sensor. Thus, the implant plug can allow the implant stem with a cavity to be inserted into a patient without the sensor.

The system can be sent to the customer (e.g., a surgeon) with an implant plug coupled to each implant stem, or the system can include a single implant plug that can be installed into any of the implant stems. Each implant plug can be installed into any of the implant stems before the implant stems are inserted into a sizing fixture. The sizing fixture can be a safeguard to verify the implant stem selected from the system matches the size of the ream in a patient's bone. After the size of the implant stem is verified, the customer can use the sizing fixture to couple one of the implant stems to a second implant component (e.g., a humeral head adapter, or any other component that can be attached to the modular implant stem). The sizing and impaction of the modular implant stem can introduce stress and pressure to implant stems. Further, the end of the implant stem can contact the sizing fixture during the sizing of the implant stem and the impaction of the second implant component to the implant stem. Thus, the implant plugs can be installed in the implant stems before the sizing and impaction process to prevent the sensor from being damaged and prevent the deformation of implant stems.

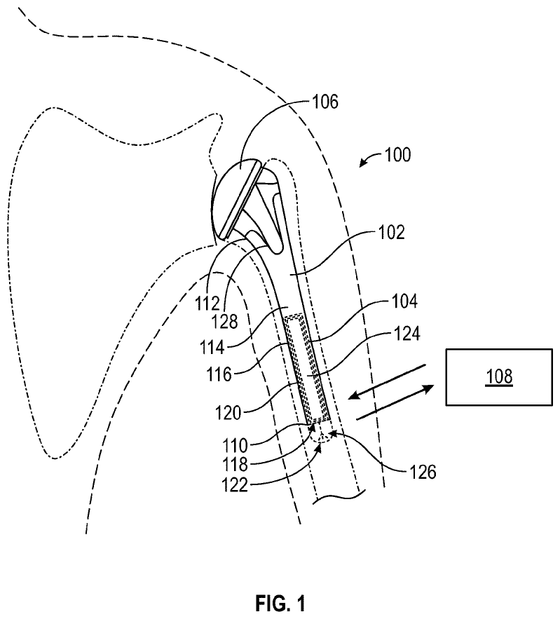

After the second implant component is coupled to the implant stem, the implant plug can be removed from the implant stem and the sensor assembly can be coupled to the implant stem. The implant stem with the sensor assembly can be installed into a reamed-out bone of the patient. Once installed in the patient, the external controller can be used to turn on the sensor of the sensor assembly and the sensor can be used to measure or detect various parameters regarding the patient's motion. In examples, the parameters captured by the sensors can be analyzed (e.g., by a doctor, nurse, or by software) to ensure the patient is achieving fitness goals such as desired range of motion for the shoulder. In examples, the parameters captured by the sensors can aid the doctor in prescribing physical therapy to help the patient achieve range of motion goals of their shoulder and can be used proactively to diagnose ailments of the shoulder. The system that includes the implant stems, the implant plugs, and the sensor assemblies will be discussed below with reference to FIGS. 1-17.

The above discussion is intended to provide an overview of subject matter of the present patent application. It is not intended to provide an exclusive or exhaustive explanation of the invention. The description below is included to provide further information about the present patent application.

FIG. 1 illustrates a schematic diagram of a smart implant 100 installed within a human body. The smart implant 100 can include an implant stem 102, a sensor assembly 104, a second implant component 106, and a controller 108. The implant stem 102 can include a first end 110 and a second end 112. An elongated body 114 can extend between the first end 110 and the second end 112. The elongated body 114 can include a cavity 116 formed therein. The elongated body 114 can extend from the first end 110 of the implant stem 102 toward the second end 112 of the implant stem 102. The cavity 116 can form an opening 118 at the first end 110 of the implant stem 102.

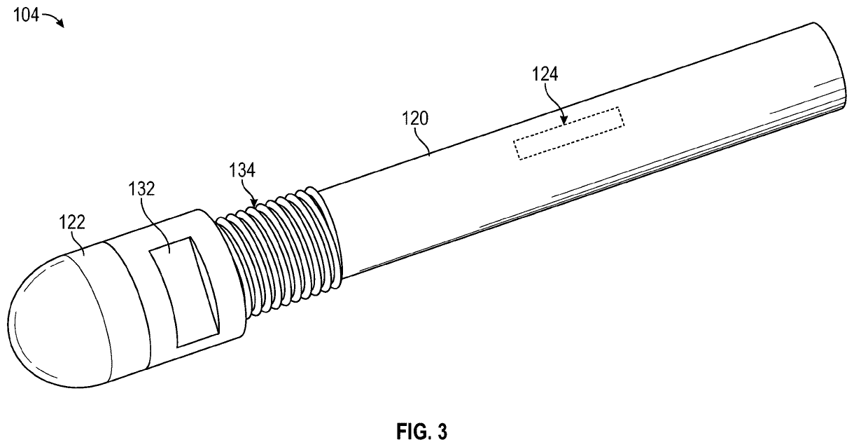

The sensor assembly 104 can be configured to fit within the cavity 116 of the implant stem 102. The sensor assembly 104 can include a sensor 124. The sensor assembly 104 can also include an antenna 126 installed within the nose cone 122. The antenna 126 can be in electrical communication with the sensor 124. The sensor assembly 104 will be discussed in more detail below with reference to FIG. 3.

The controller 108 can include one or more processors, microprocessors, microcontrollers, electronic control modules (ECMs), electronic control units (ECUs), programmable logic controller (PLC), or any other suitable means for electronically communicating with smart implant 100. The controller 108 can be configured to operate according to a predetermined algorithm or set of instructions for communicating with smart implant 100. Such an algorithm or set of instructions can be stored in a database, can be read into an on-board memory of the controller 108, or preprogrammed onto a storage medium or memory accessible by the controller 108, for example, in the form of a floppy disk, hard drive, optical medium, random access memory (RAM), read-only memory (ROM), or any other suitable computer-readable storage medium commonly used in the art (each referred to as a “database”), which can be in the form of a physical, non-transitory storage medium.

The controller 108 can be in electrical communication or connected to a display (not shown), or the like, and various other components, or multiple smart implants, like smart implant 100. By way of such connection, the controller 108 can receive data pertaining to the rehabilitation or diagnostic data stored within the sensor assembly 104. In response to such input, the controller 108 can perform various determinations and transmit output signals corresponding to the results of such determinations or corresponding to actions that need to be performed, such as alerting the doctor of any recommended physical therapy exercises or alerting the doctor to any potential diagnoses.

The controller 108, including a human-machine interface, can include various output devices, such as screens, video displays, monitors and the like that can be used to display information, warnings, data, such as text, numbers, graphics, icons, and the like, regarding the status of the smart implant 100. The controller 108, including the human-machine interface, can additionally include a plurality of input interfaces for receiving information and command signals from various sensors associated with the smart implant 100 and a plurality of output interfaces for sending control signals to smart implant 100. Suitably programmed, the controller 108 can serve many additional similar or wholly disparate.

The second end 112 of the implant stem 102 can include an attachment mechanism 128. The attachment mechanism 128 can be configured to receive any of the various different types of the second implant component 106. For example, as shown in FIG. 1, the second implant component 106 can be a humeral head adapter configured to interact with opposing implant components (e.g., a glenoid component). In another example, the second implant component 106 can be a humeral cap configured to interact with opposing implant components (e.g., a glenosphere). In yet another example, second implant component 106 can be a component used in knee, hip, or any other joint replacement configured to interact with their respective opposing implant components.

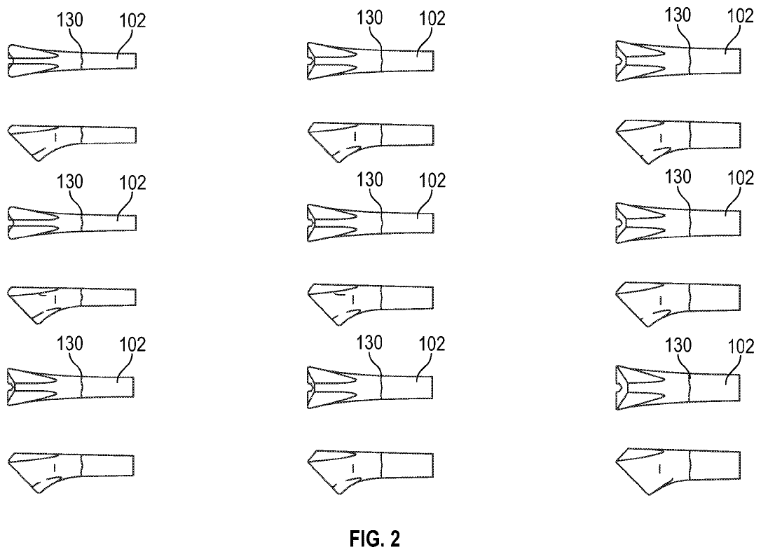

FIG. 2 illustrates a schematic diagram of a plurality of the implant stem 102. As shown in FIG. 2, each of the implant stem 102 of the plurality, can have an elongated body diameter 130. The elongated body diameter 130 of the implant stem 102 can range from 4.0 millimeters-20.0 millimeters (0.157 inches-0.787 inches). The elongated body diameter 130 can be selected to match a reamed-out portion of a bone of the patient.

FIG. 3 illustrates a perspective view of an example of the sensor assembly 104. The sensor assembly 104 can include a sensor housing 120, a nose cone 122, and at least one sensor 124. The at least one sensor 124 can include a combination of different types of sensors (e.g., an accelerometer, a gyro sensor, a piezoelectric sensor, thermometer, strain gauge, or any combination thereof, including any other sensor that can be used to detect motion within a body) that can log, and store information. As shown in the example shown in FIG. 1, the sensor 124 can be within the sensor housing 120. The sensor 124 can be configured to fit within the cavity 116 (FIG. 1) of the implant stem 102 (FIG. 1).

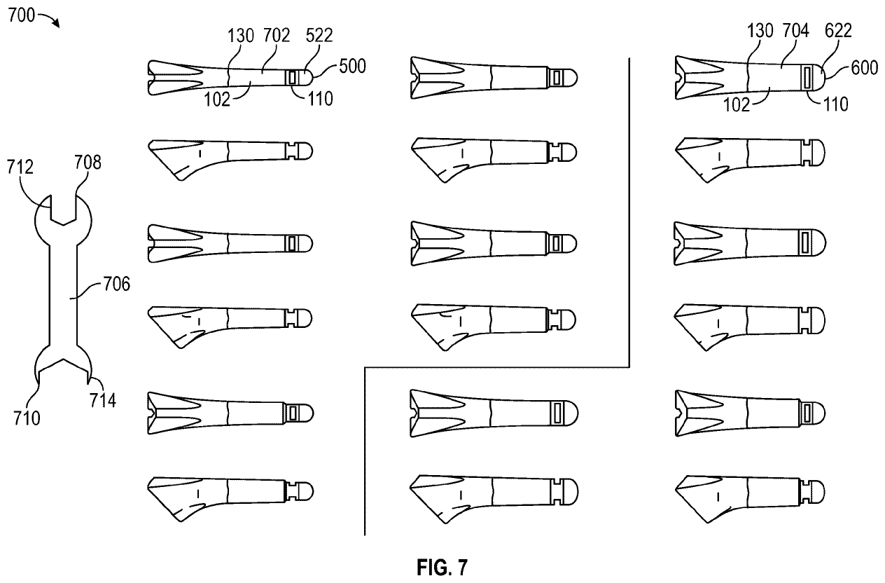

In one example, the nose cone 122 can include a mechanical interface 132 and threads 134. In another example, the nose cone 122 can include a mechanical interface 132, and the sensor housing 120 can include the threads 134. A tool (e.g., a tool 706 first shown in FIG. 7) can engage the mechanical interface 132 to couple the sensor assembly 104 and the implant stem 102 or to decouple the sensor assembly 104 and the implant stem 102. The mechanical interface 132 will be discussed in more detail below.

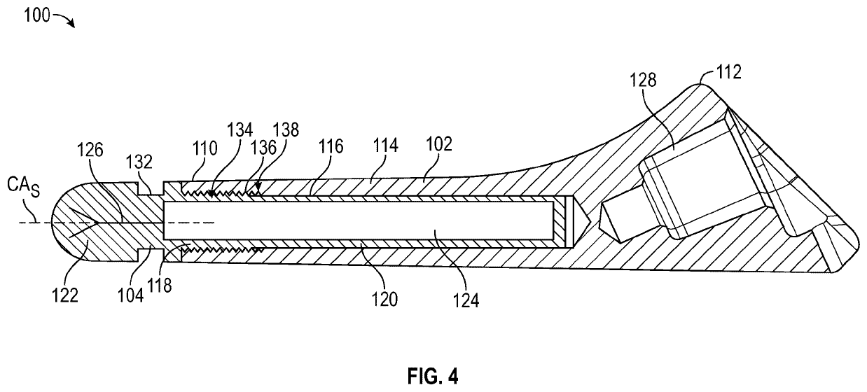

FIG. 4 illustrates a schematic diagram of a cross-sectional view of an example of the sensor assembly 104 installed in the implant stem 102. A portion of the sensor assembly 104 can be configured to fit within the cavity 116 of the elongated body 114. When installed in the first end 110 of the implant stem 102, the nose cone 122 of the sensor assembly 104 can form an end of the implant stem 102.

The cavity 116 of the implant stem 102 can include an inner surface 136. The inner surface 136 can include threads 138. The sensor assembly 104 can also include the threads 134 (e.g., formed on the sensor housing 120, the nose cone 122, or on the sensor 124). The threads 138 of the implant stem 102 can be complementary to the threads 134 of the sensor assembly 104. Thus, the threads 138 of the implant stem 102 and the threads 134 of the sensor assembly 104 can couple the implant stem 102 and the sensor assembly 104.

For example, the tool can engage the mechanical interface 132 of the sensor assembly 104 to rotate the sensor assembly 104 clockwise about a central axis CAs and engage the threads 134 of sensor assembly 104 and the threads 138 of the implant stem 102 to couple the sensor assembly 104 and the implant stem 102. The tool can also engage the mechanical interface 132 of the sensor assembly 104 to rotate the sensor assembly 104 counterclockwise about the central axis CAs to decouple the sensor assembly 104 and the implant stem 102.

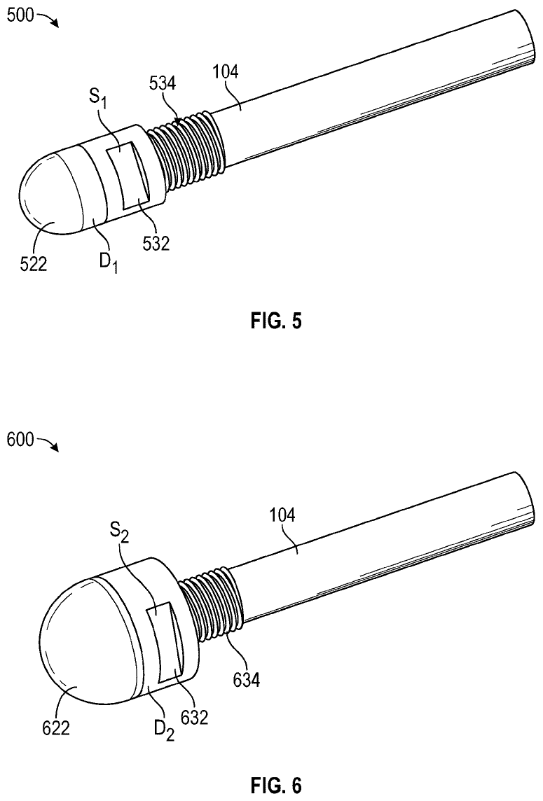

FIGS. 5 and 6 will be discussed together. FIG. 5 illustrates a schematic diagram of a first sensor assembly 500. FIG. 6 illustrates a schematic diagram of a second sensor assembly 600. The first sensor assembly 500 can be, for example, the sensor assembly 104 (FIGS. 1 and 4), or any other sensor that can be inserted into the implant stem 102 (FIGS. 1, 2, and 4) to create the smart implant 100 (FIG. 1). A nose cone 522 of the first sensor assembly 500 can have a diameter D₁. The nose cone 522 can include a mechanical interface 532 of a size S₁. The first sensor assembly 500 can include threads 534 and the second sensor assembly 600 can include threads 634.

The second sensor 600 can be, for example, the sensor assembly 104, or any other sensor that can be inserted into a larger stock size of the implant stem 102 to create the smart implant 100. A nose cone 622 of the second sensor assembly 600 can have a diameter D₂. The nose cone 622 can include a mechanical interface 632 of a size S₂.

In a first example, the diameter D₁ of the nose cone 522 can be smaller than the diameter D₂ of the nose cone 622. In a second example, the diameter D₁ of the nose cone 522 can be larger than the diameter D₂ of the nose cone 622. In the first example, the size S₁ of the mechanical interface 532 can be smaller than the S₂ of the mechanical interface 632. In the second example, the size S₁ of the mechanical interface 532 can be larger than the size S₂ of the mechanical interface 632.

FIG. 7 illustrates a schematic diagram of a system 700 including a first plurality of stems 702 and a second plurality of stems 704. The first plurality of stems 702 can be a plurality of implant stems (e.g., can include implant stem 102). The second plurality of stems 704 can be a plurality of implant stems (e.g., can include implant stem 102). The elongated body diameter 130 of each of the implant stem 102 of the first plurality of stems 702 can be smaller than the elongated body diameter 130 of the implant stem 102 of the second plurality of stems 702. The first plurality of stems 702 can have different stock sizes from the second plurality of stems 704 and can be generally smaller including in diameter. This will be discussed in more detail below.

As shown in FIG. 7, the first plurality of stems 702 can be configured to receive the first sensor assembly 500. The elongated body diameter 130 of the implant stem 102 of the first plurality of stems 702 can be similar to the diameter D₁ of the nose cone 522 of the first sensor assembly 500. Because the elongated body diameter 130 of the implant stem 102 of the first plurality of stems 702 and the diameter D₁ of the nose cone 522 of the first sensor assembly 500 can be similar, there is only a minimal step at the transition from the nose cone 522 to the first end 110 of the implant stem 102 when the first sensor assembly 500 is installed in any of the implant stem 102 of the first plurality of stems 702.

The second plurality of stems 704 can be configured to receive the second sensor assembly 600. The elongated body diameter 130 of the implant stem 102 of the second plurality of stems 704 can be similar to the diameter D₂ of the nose cone 622 of the second sensor assembly 600. Because the elongated body diameter 130 of the implant stem 102 of the second plurality of stems 704 and the diameter D₂ of the nose cone 622 of the second sensor assembly 600 can be similar, there is only a minimal step at the transition from the nose cone 622 to the first end 110 of the implant stem 102 when the second sensor assembly 600 is installed in any of the implant stem 102 of the second plurality of stems 704.

The system 700 can include a tool 706 having a first end 708 opposite a second end 710. The first end 708 can include a first mechanical interface 712. The first end 708 can be configured to engage a mechanical interface (e.g., mechanical interface 532) that is size S₁. Thus, the first end 708 of the tool 706 can be used to couple or decouple the first sensor assembly 500 and implant stem 102 of the first plurality of stems 702.

The second end 710 can include a second mechanical interface 714. The second end 710 can be configured to engage a mechanical interface (e.g., mechanical interface 632) that is size S₂. Thus, the second end 710 of the tool 706 can be used to couple or decouple the second sensor assembly 600 and implant stem 102 of the second plurality of stems 704.

As shown in FIG. 7, the system 700 can include the first sensor assembly 500 and the second sensor assembly 600. In another example, the system 700 can include either the first sensor assembly 500 or the second sensor assembly 600. In examples, the system 700 can include only the first sensor assembly 500. However, if the system 700 only includes the first sensor assembly 500 there can be a large step between the nose cone 522 of the first sensor assembly 500 and the elongated body diameter 130 of the implant stem 102 of the second plurality of stems 704.

In other examples, the system 700 can include only the second sensor assembly 600. However, if the system 700 only includes the second sensor assembly 600 there can be a large step between the nose cone 622 of the second sensor assembly 600 and the elongated body diameter 130 of the implant stem 102 of the first plurality of stems 702.

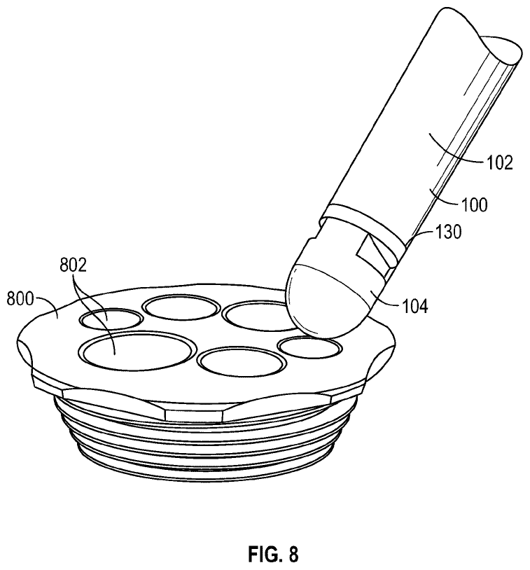

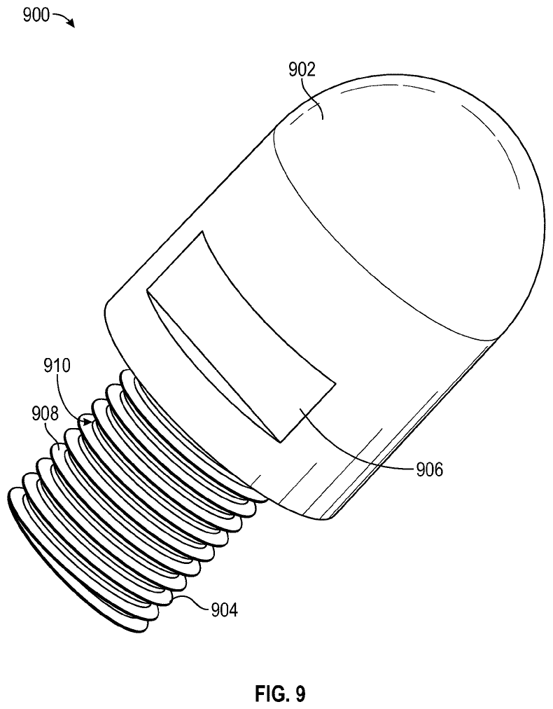

FIG. 8 illustrates a schematic diagram of the smart implant 100 and a sizing fixture 800. The sizing fixture 800 includes a plurality of sizing holes (hereinafter “sizing holes 802”). Each of the sizing holes 802 can correspond to at least one of the elongated body diameter 130 of any of the implant stem 102 of the system 700 (FIG. 7). Each of the sizing holes 802 can be configured to receive the elongated body 114 of at least one of the implant stems 102 of the system 700.

The sizing fixture 800 can be configured to hold the implant stem 102 while the second implant component 106 (FIG. 1) is impacted into the attachment mechanism 128 (FIG. 1) on the second end 112 (FIG. 1) of the implant stem 102. The impaction of the second implant component 106 and the attachment mechanism 128 of the implant stem 102 can expose the implant stem 102 to stress. Moreover, if the sensor assembly 104 is installed in the implant stem 102 during the impaction process, the sensor assembly 104 can be damaged.

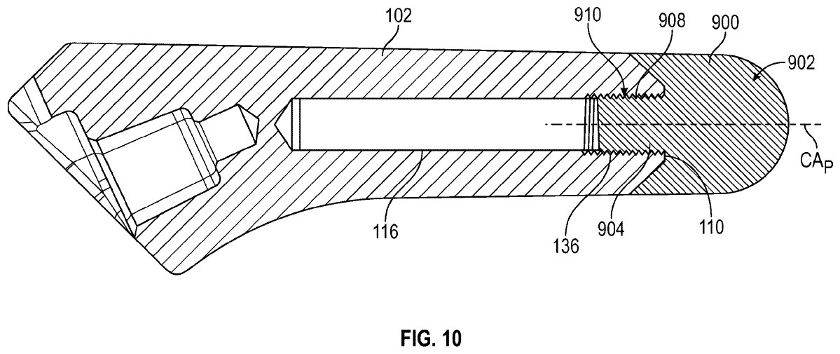

FIG. 9 illustrates a perspective view of an implant plug 900. FIG. 10 illustrates a cross-sectional view of an example of the implant plug 900 from FIG. 9 installed in an implant stem 102. FIGS. 9 and 10 will be discussed together.

The implant plug 900 can include a nose cone 902 and a rod 904. The nose cone 902 of the implant plug 900 can include a mechanical interface 906. The rod 904 can extend from the nose cone 902. The rod 904 of the implant plug 900 can be configured to fit within the cavity 116 of the implant stem 102.

The mechanical interface 906 can be configured to be engaged by the tool 706 (FIG. 7). The mechanical interface 906 can be either size S₁ or size S₂. The mechanical interface 906 can be configured to rotate the implant plug 900 about its central axis CAP when engaged with a tool (e.g., tool 706). As discussed above, the inner surface 136 of the implant stem 102 can include threads 138. As shown in FIG. 9, the rod 904 can include an outer surface 908 having a threads 910 formed thereon. The threads 910 on the rod 904 of the implant plug 900 can be complementary to the threads 138 on the inner surface 136 of the implant stem 102. Thus, the implant plug 900 can be coupled to and decoupled from the implant stem 102. When the implant plug 900 is coupled to the implant stem 102 the nose cone 902 can form an end of the implant stem 102.

As discussed above, the sizing fixture 800 (FIG. 8) can be configured to hold the implant stem 102 while the second implant component 106 (FIG. 1) is impacted into the attachment mechanism 128 (FIG. 1) on the second end 112 (FIG. 1) of the implant stem 102. The impaction of the second implant component 106 and the attachment mechanism 128 of the implant stem 102 can expose the implant stem 102 to stress. As shown in FIG. 10, the implant plug 900 can be coupled to the implant stem 102. A tool (e.g., tool 706 (FIG. 7)) can be used to engage with the mechanical interface 906 of the implant plug 900. The tool can rotate the implant plug 900 clockwise around the center axis CAP such that the threads 910 on the rod 904 engage with the threads 138 on the inner surface 136 of the implant stem 102 and the implant plug 900 can be coupled to the implant stem 102. When the implant plug 900 is coupled to the implant stem 102 the implant plug 900 can help protect the implant stem 102 during the sizing and impaction within sizing fixture 800. After impaction, the tool 706 can be used to engage with the mechanical interface 906 of the implant plug 900 to rotate the implant plug 900 counterclockwise about the center axis CAP of the implant plug 900 to decouple the implant plug implant plug 900 from the implant stem 102.

In examples, the implant plug 900 can be made of titanium, steel, copper, nickel, carbon fiber, polymers, and any alloys or composites thereof, or any other material that can be machined to form an implant plug.

In one example, the system 700 can include one of the implant plug 900 that can be coupled to any of the implant stem 102 of the system 700. In another example, the system 700 can include one of the implant plug 900 coupled to each of the implant stem 102 of the system 700. When the implant plug 900 is coupled to the implant stem 102 the implant plug 900 can retain the shape of an implant stem that does not include a cavity. Thus, the implant plug 900 can be installed in the implant stem 102 to enable the implant stem 102 to be inserted in a human body without the sensor assembly 104.

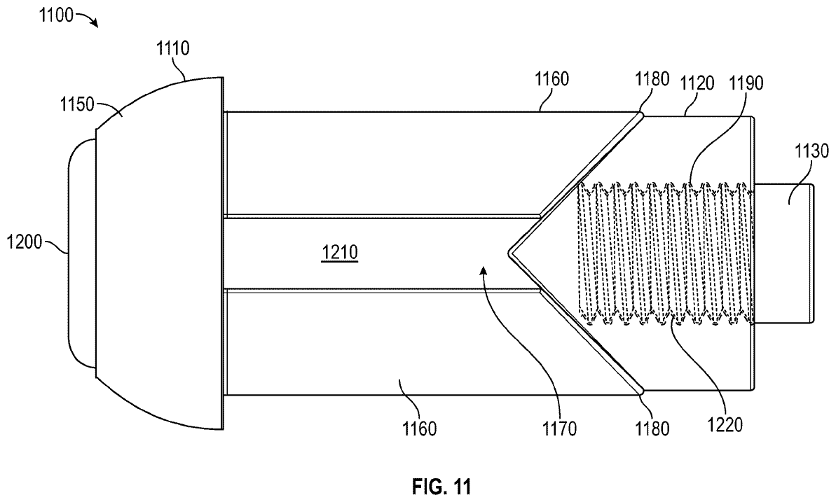

FIG. 11 illustrates an example of an implant plug 1100. The implant plug 1100 can be insertable into a humeral stem (e.g., the implant stem 102 from FIG. 1) to protect the humeral stem during impaction of the stem with a second implant component (e.g., the second implant component 106 from FIG. 1). The implant plug 1100 can include an expander plug 1110, an expander wedge 1120 and an expander screw 1130.

The expander plug 1110. In examples, the expander plug 1110 can be made from titanium, stainless steel, ceramic, any other biocompatible material, or the like. The expander plug 1110 can include a hollow head 1150 and at least two expansion walls (expansion walls 1160). In examples, the expander plug 1110 can be a monolithic component. In another example, the expander plug 1110 can be a composite with the hollow head 1150 made of a first material and the expansion walls 1160 made of a second material.

The hollow head 1150 can define a portion of a channel 1170. The channel 1170 can be within the implant plug 1100. The hollow head 1150 can have a diameter larger than any other portion of the implant plug 1100 such as the hollow head 1150 engages with the humeral stem when the implant plug 1100 is installed within the cavity of the humeral stem. In examples, an outer contour of the hollow head 1150 can match a curvature of the humeral stem such that there is no change of diameter between a portion of the hollow head 1150 that is adjacent to the humeral stem and the humeral stem adjacent to the installed expander plug 1110.

The expansion walls 1160 can be configured to expand and retract to change a diameter of the implant plug 1100. The expansion walls 1160 can retract to enable insertion or removal of the implant plug 1100 into the cavity of the humeral stem. The expansion walls 1160 can expand to couple the implant plug 1100 to the humeral stem. The expansion walls 1160 can include a distal portion 1180. In an example, the expansion walls 1160 can have a resting position that is less than a cavity of the humeral stem such that the expansion walls 1160 have clearance within the cavity of the humeral stem. Thus, the expansion walls 1160 can return to the resting position after the expander wedge 1120 is removed from the expander plug 1110.

The expander wedge 1120 can be configured to engage with the distal portion 1180 of the expansion walls 1160 to expand or retract the expansion walls 1160. As shown in FIG. 11, the expander wedge 1120 can be a cylindrical body with a wedge formed on an end. The expander wedge 1120 can define a portion of the channel 1170. In examples, the expander wedge 1120 can include a threaded internal surface 1190 (shown in phantom).

The expander screw 1130 can be configured to extend within the channel 1170 to actuate the implant plug 1100 to expand or retract the expander plug 1110 by interacting with the expander wedge 1120. In examples, the expander screw 1130 can include a head 1200 and a stem 1210. The head 1200 can have a wider diameter than the stem 1210 such that the head 1200 engages with the hollow head 1150 when the expander screw 1130 is inserted into the channel 1170. The stem 1210 can include a threaded surface 1220 (shown in phantom).

In an operational example, the threaded internal surface 1190 of the expander wedge 1120 and the threaded surface 1220 on the stem 1210 of the expander screw 1130 are complementary such that the threads (e.g., the threaded internal surface 1190 and the threaded surface 1220) will pull the expander wedge 1120 toward the head 1200 of the expander screw 1130 and the expander wedge 1120 can engage with the distal portion 1180 of the at least two expansion walls 1160 to move the at least two expansion walls 1160 radially outward, on condition that the expander screw 1130 is rotated clockwise.

In another operational example, the threaded internal surface 1190 of the expander wedge 1120 and the threaded surface 1220 on the stem 1210 of the expander screw 1130 are complementary such that the threads (e.g., the threaded internal surface 1190 and the threaded surface 1220) will push the expander wedge 1120 away from the head 1200 of the expander screw 1130 and the expander wedge 1120 will disengage with the distal portion 1180 of the at least two expansion walls 1160 to move the at least two expansion walls 1160 radially inward, on condition that the expander screw 1130 is rotated counterclockwise.

In examples, the expander plug 1110 can be made from titanium, stainless steel, any other biocompatible material, or the like. Thus, the expander plug 1110 can be installed in a bone of a patient with the humeral stem. Here, a system (e.g., the system 100), can be delivered to a surgeon with the expander plug 1110 installed into the cavity of each of the plurality of stems. The surgeon can then decide to remove the expander plug 1110 to insert a sensor assembly into the humeral stem intraoperatively.

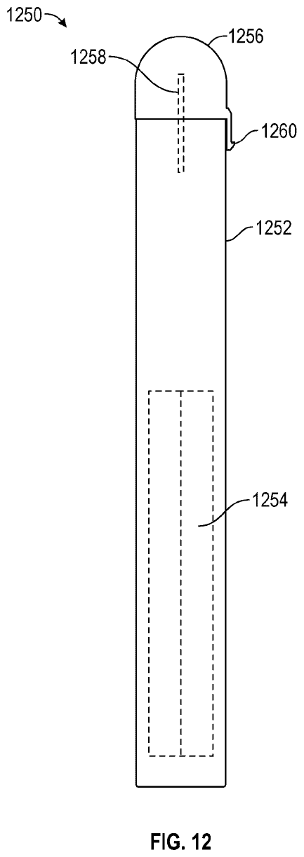

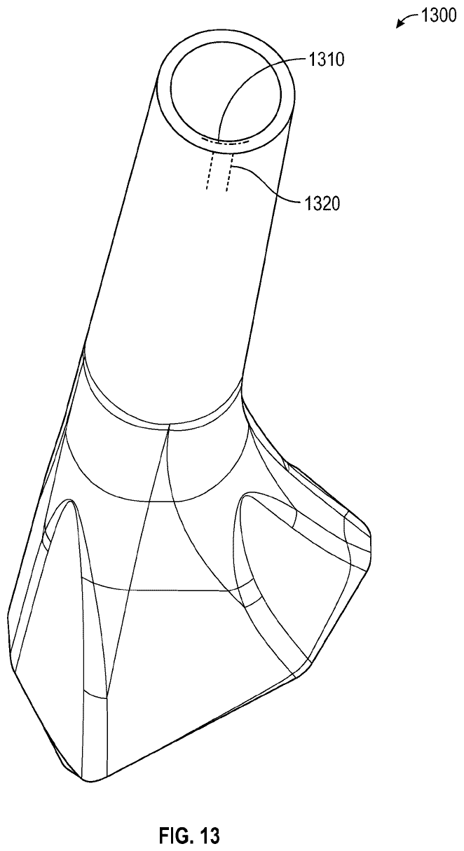

An example of a sensor assembly and a humeral stem will be discussed in FIGS. 12-14.

FIG. 12 illustrates an example of a sensor assembly 1250. The sensor assembly 1250 can include a sensor housing 1252, a sensor 1254, and an end cap 1256. The sensor assembly 1250 can fit within a cavity of a humeral stem to detect motion of an implant after the humeral stem is installed within a shoulder of a patient.

The sensor housing 1252 can be sized such that the sensor housing fits within a cavity of a humeral stem. The sensor housing 1252 can be made from titanium, stainless steel, any other biocompatible material, or the like. The sensor housing 1252 can protect the sensor 1254 and can provide support to the cavity of the humeral stem such as to prevent collapse of the humeral stem.

The sensor 1254 can be installed within the sensor housing 1252. In another example, the sensor 1254 can form the sensor housing 1252 such that an exterior of the sensor 1254 is the sensor housing 1252.

The end cap 1256 can attach to the sensor housing 1252. In examples, the end cap 1256 can be removably attached to the sensor housing 1252. When installed on the sensor housing 1252, the end cap 1256 can form an end of the the sensor assembly 1250. The end cap 1256 can include an antenna 1258 and a coupling interface 1260.

The antenna 1258 can communicatively connect the sensor 1254 with a controller. The antenna 1258 can be integral the end cap 1256. In examples, the antenna 1258 can be within the end cap 1256. In another example, the antenna 1258 can be installed in a location other than the end cap 1256. Here, the antenna 1258 can be installed within the sensor housing 1252.

The coupling interface 1260 can interact with a coupling receptacle of the cavity of the humeral stem. As shown in FIG. 12, the coupling interface 1260 can be a tab that extends radially outward from the sensor housing 1252. In another example, the coupling interface 1260 can extend radially outward from the end cap 1256. The coupling interface 1260 can be a post, dome, any other protrusion, any other engageable shape that can engage with the coupling receptacle of the humeral stem to hold the sensor assembly 1250 within the cavity of the humeral stem, or the like. The coupling interface 1260 can be located around the sensor housing 1252 or the end cap 1256 such that the sensor 1254 is positioned in a specific location within the cavity of the humeral step when the coupling interface 1260 is engaged with the coupling receptacle of the humeral stem.

FIG. 13 illustrates another example of one of a plurality of stems (e.g., the implant stem 102, hereinafter referred to as stem 1300). Stem 1300 can include a coupling receptacle 1310 and an alignment indicator 1320.

The coupling receptacle 1310 can be engageable with the coupling interface 1260 (FIG. 12) to hold the sensor assembly (e.g., the sensor assembly 1250) within any of the stem 1300. The coupling receptacle 1310 can be a cavity, or any other coupling feature that can receive or engage with the coupling interface 1260. In examples, the coupling receptacle 1310 can ensure special alignment of the sensor within the humeral stem. For example, the coupling receptacle 1310 can maintain an orientation of the sensor assembly within the humeral stem to create special alignment between the data from the various sensors within the humeral stem. The various sensors within the humeral stem can be gyroscopes, accelerometers, or the like. Therefore, the coupling receptacle 1310 can be configured to position the sensors within the humeral stem to control the orientation of the sensors when the implant is installed within a bone of the patient.

The alignment indicator 1320 can indicate a location of the coupling receptacle 1310 to aid in the attachment of the sensor assembly and the stem 1300. The alignment indicator 1320 can be formed on an external surface of the stem 1300. In another example, the alignment indicator 1320 can be a marking or design on the external surface of the stem 1300. In another example, the stem 1300 can include a poka-yoke design to prevent the improper installation of the sensor assembly. For example, an opening of a cavity of the stem 1300 can include a cut-out that permits the coupling interface 1260 to slide within the cavity and engage with the coupling receptacle 1310.

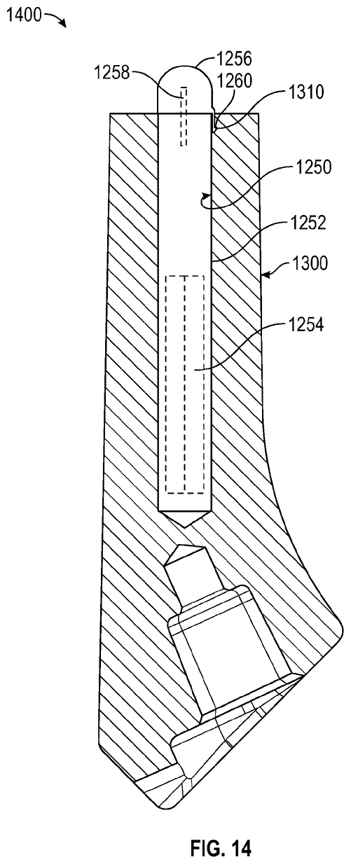

FIG. 14 illustrates a cross-sectional view of an implant 1400. The implant 1400 can include a sensor assembly 1250 and a stem 1300. The sensor assembly 1250 can be installed within the stem 1300 such that the end cap 1256 extends outside of the stem 1300. Here, the end cap 1256 can form an end of the stem 1300. In another example, the sensor assembly 1250 can be installed completely within the stem 1300.

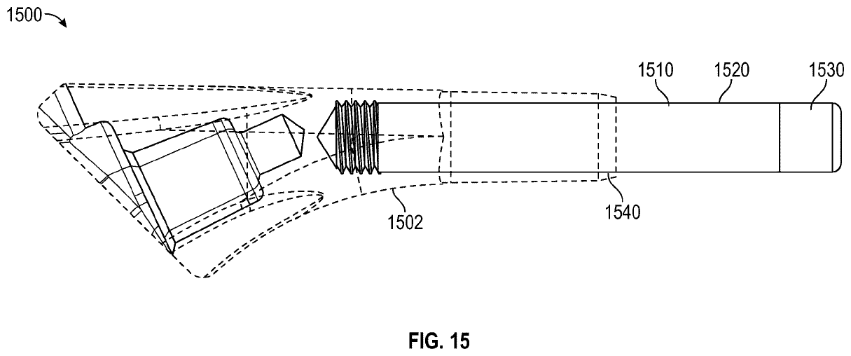

FIG. 15 illustrates an example of an implant 1500. The implant 1500 can include a stem 1502 and a sensor assembly 1510. The sensor assembly 1510 can be installed within the stem 1502. For example, the sensor assembly 1510 can be partially installed such that a portion of a sensor housing 1520 and an end cap 1530 extend outside the stem 1502. Here, the sensor housing 1520 together with the stem 1502 forms part of a shaft of the smart implant 100. Thus, the stem 1502 length can be reduced using the sensor housing 1520 as part of the implant 1500 interfacing with the bone of a patient. In an example, a portion of the sensor housing 1520 can face a portion of the bone rather than being received within the stem 1502.

To support the sensor assembly 1510, the stem 1502 can include a tapered edge 1540 that extends axially to support the sensor assembly 1510 within the stem 1502. The additional support provided by the tapered edge 1540 helps support the sensor assembly 1510 when the implant 1500 is installed within a bone of a patient. For example, the implant 1500 can be installed within a reamed portion of a humerus, femur, or the like.

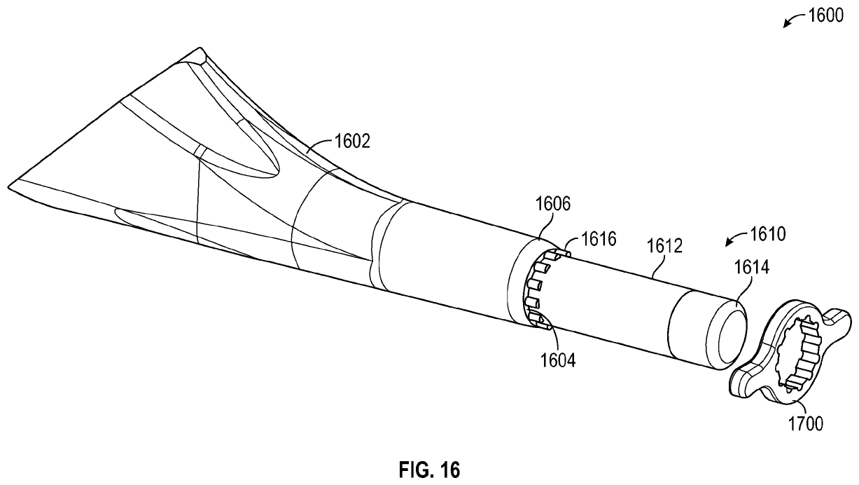

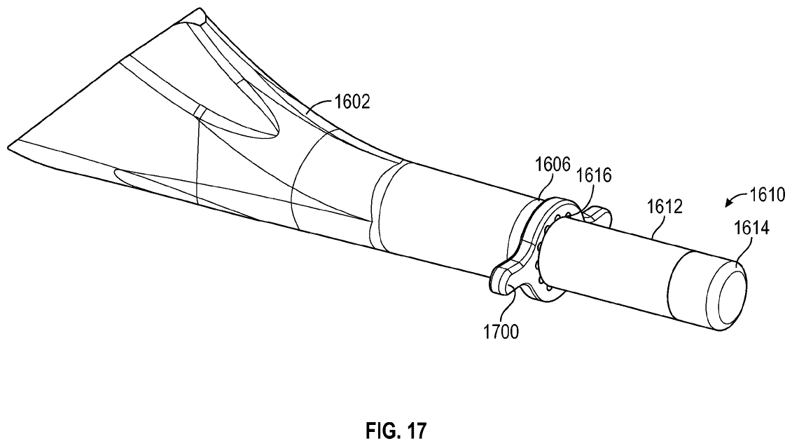

FIGS. 16 and 17 will be discussed together below. FIG. 16 illustrates another example of an implant 1600. FIG. 17 illustrates an example of the implant 1600 with a tool 1700 engaged. The implant 1600 can include a stem 1602 and a sensor assembly 1610. The stem 1602 can include a cavity 1604 formed therein. The stem 1602 can also include a tapered portion 1606 formed on a distal portion of the cavity 1604. The cavity 1604 can receive the sensor assembly 1610. The sensor assembly 1610 can include a sensor housing 1612 and an end cap 1614. The sensor housing 1612 can include protrusions 1616 extending radially outward from the sensor housing 1612.

The sensor assembly 1610 can be installed in the stem 1602 such that at least a portion of the sensor housing 1612 and the end cap 1614 extend axially outside the stem 1602. The protrusions 1616 can be positioned on the sensor housing 1612 such that the protrusions 1616 contact the tapered portion 1606 when the sensor assembly 1610 is installed within the cavity 1604 of the stem 1602. The protrusions 1616 help distribute forces from the sensor assembly 1610 to the stem 1602. Thus, the protrusions 1616 help protect the sensor housing 1612 from stress during the installation of the implant 1600 into a bone of the patient.

As discussed above, a surface of the stem 1602 and a surface of the sensor assembly 1610 can include complementary threads such that the sensor assembly 1610 can be removably attached to the stem 1602. In the example shown in FIG. 17, the threads can be configured such that clockwise rotation of the sensor assembly 1610 within the stem 1602 can removably couple the sensor assembly 1610 and the stem 1602. In another example, the threads can be configured such that counterclockwise rotation of the sensor assembly 1610 within the stem 1602 can removably couple the sensor assembly 1610 and the stem 1602. In yet another example, another removable coupling mechanism can be used as an interface between the stem 1602 and the sensor assembly 1610. For example, a snap ring, pin, set screw, or the like can be utilized to secure the sensor assembly 1610 within the stem 1602. In examples, the tool 1700 can be used to engage with the protrusions 1616 to install or remove the sensor assembly 1610 from the stem 1602. As shown in FIGS. 16 and 17, the tool 1700 can have cut outs to receive the protrusions 1616 and can engage with the protrusions 1616 upon rotation of the tool 1700.

### NOTES AND EXAMPLES

The following, non-limiting examples, detail certain aspects of the present subject matter to solve the challenges and provide the benefits discussed herein, among others.

Example 1 is a system configured to be implanted into a human body, the system comprising: a plurality of stems, each of the plurality of stems having different sizes from one another and are configured for insertion into a bone of a patient, each of the plurality of stems having an elongated body containing a cavity having an opening at an end thereof; and a sensor assembly configured to removably couple to one or more of the plurality of stems, and the sensor assembly includes: a sensor configured for insertion within the cavity; and a nose cone extending from the sensor and projecting from the cavity and covers the opening when a sensory assembly is coupled with one of the plurality of stems.

In Example 2, the subject matter of Example 1 optionally includes wherein each of the plurality of stems comprises an attachment mechanism formed in the elongated body opposite the cavity, and wherein the attachment mechanism is configured to receive a second implant component.

In Example 3, the subject matter of Example 2 optionally includes wherein the cavity of each of the plurality of stems further comprises an internal surface having threads formed thereon adjacent to the opening of the cavity of each of the plurality of stems.

In Example 4, the subject matter of Example 3 optionally includes wherein the sensor assembly includes: a sensor housing having the sensor therein, the sensor housing is configured to be inserted within the cavity of the elongated body of one or more of the plurality of stems.

In Example 5, the subject matter of Example 4 optionally includes wherein the sensor housing includes an exterior surface having threads, the threads of the exterior surface of the sensor assembly are complementary to the threads formed on the internal surface of the cavity of the plurality of stems so that the sensor assembly is configured to be screwed into the cavity of one or more of the plurality of stems.

In Example 6, the subject matter of Example 5 optionally includes wherein the system includes a first sensor assembly of a first size and a second sensor assembly of a second size, and wherein the nose cone of the first sensor assembly has a first diameter and the nose cone of the second sensor assembly has a second diameter.

In Example 7, the subject matter of Example 6 optionally includes a first group of the plurality of stems; and a second group of the plurality of stems, wherein each stem of the first group of the plurality of stems has a smaller external diameter than each stem of the second group of the plurality of stems, wherein at least the first sensor assembly is configured to couple with the first group of the plurality of stems and at least the second sensor assembly is configured to couple with the second group of the plurality of stems.

In Example 8, the subject matter of Example 7 optionally includes an implant plug including: a nose cone; and a rod extending from the nose cone, the rod has threads formed on an outer surface that are complementary to the threads formed on the internal surface of the cavity of each of the plurality of stems, wherein the implant plug forms an end of each of the plurality of stems when installed in each of the plurality of stems, and wherein the implant plug is configured to provide structural support during assembly of the second implant component and each of the plurality of stems when installed in each of the plurality of stems.

In Example 9, the subject matter of Example 8 optionally includes a sizing fixture having a plurality of holes, each hole of the plurality of holes corresponds to at least one of external diameters of the elongated body of at least one of the plurality of stems, wherein each hole of the plurality of holes in the sizing fixture is configured to receive the elongated body of at least one of the plurality of stems and hold the at least one of the plurality of stems while the second implant component is impacted into the attachment mechanism.

In Example 10, the subject matter of any one or more of Examples 1-9 optionally include an implant plug assembly including: an expander plug having: a hollow head defining a portion of a channel within the implant plug assembly; expansion walls extending from the hollow head and including a distal portion; an expander wedge defining a portion of the channel within the implant plug assembly, the expander wedge having a threaded internal surface; and an expander screw having: a head; and a stem extending from the head, the stem extending through the channel within the implant plug assembly, and including a threaded surface.

In Example 11, the subject matter of Example 10 optionally includes wherein the threaded internal surface of the expander wedge and the threaded surface on the stem of the expander screw are complementary such that the threaded surfaces will pull the expander wedge toward the head of the expander screw and the expander wedge will engage with the distal portion of the expansion walls to move the expansion walls radially outward, on condition that the expander screw is rotated clockwise.

In Example 12, the subject matter of any one or more of Examples 10-11 optionally include wherein the threaded internal surface of the expander wedge and the threaded surface on the stem of the expander screw are complementary such that the threaded surfaces will push the expander wedge away from the head of the expander screw and the expander wedge will disengage with the distal portion of the expansion walls to move the expansion walls radially inward, on condition that the expander screw is rotated counterclockwise.

In Example 13, the subject matter of any one or more of Examples 1-12 optionally include wherein the sensor assembly includes: a sensor housing having a coupling interface; and a sensor installed at least partially within the sensor housing, wherein: the cavity of each humeral stem includes a coupling receptacle complementary to the coupling interface of the sensor housing so that the sensor assembly can be removably attached to each of the humeral stem; and the sensor housing forms an end of the implant when the sensor housing is coupled to the humeral stem.

In Example 14, the subject matter of Example 13 optionally includes wherein the humeral stem comprises an alignment marking indicative of a location of the coupling receptacle.

In Example 15, the subject matter of any one or more of Examples 1-14 optionally include an antenna within the nose cone, wherein the antenna is in electric communication with the sensor.

In Example 16, the subject matter of any one or more of Examples 3-15 optionally include an implant plug including: a nose cone; and a rod extending from the nose cone, the rod has threads formed on an outer surface that are complementary to the threads formed on the internal surface of the cavity of each of the plurality of stems, wherein the implant plug forms an end of each of the plurality of stems when installed in each of the plurality of stems, and wherein the implant plug is configured to provide structural support during assembly of the second implant component and each of the plurality of stems when installed in each of the plurality of stems; wherein the nose cone of the implant plug and the nose cone of the sensor assembly each include a mechanical interface configured to help rotate the implant plug and the sensor assembly about their central axes to install or remove the implant plug or the sensor assembly within any one of the plurality of stems.

In Example 17, the subject matter of Example 16 optionally includes a tool configured to engage with the mechanical interface so that the implant plug and the sensor assembly can be removed and installed in the cavity extending through a distal portion of any of the plurality of stems.

Example 18 is an implant that is insertable into a shoulder of a patient, the implant comprising: a joint interface; and a humeral stem coupled to the joint interface, the humeral stem including: an elongated body with a distal portion; a cavity extending through the distal portion and into the elongated body; and an attachment mechanism on an opposing end of the humeral stem from the cavity, wherein the attachment mechanism is configured to receive the joint interface.

In Example 19, the subject matter of Example 18 optionally includes an implant plug having: a cylindrical body extending between a first end and a second end; a nose cone extending from the first end of the cylindrical body; and a shaft extending from the second end of the cylindrical body, wherein: the cavity in the humeral stem and the shaft of the implant plug have complementary threads so that the implant plug can be removably attached to the humeral stem; and the nose cone forms an end of the implant when the implant plug is coupled to the humeral stem.

In Example 20, the subject matter of Example 19 optionally includes a sensor assembly, wherein the sensor assembly comprises: a sensor housing; a nose cone extending from the sensor housing; and a sensor installed at least partially within the sensor housing, wherein: the cavity in the humeral stem and the sensor housing have complementary threads so that the sensor assembly can be removably attached to the humeral stem; and the nose cone of the sensor assembly forms an end of the implant when the sensor housing is coupled to the humeral stem.

In Example 21, the subject matter of Example 20 optionally includes wherein at least a portion of the sensor housing and the nose cone extend outside the humeral stem, and wherein the distal portion of the humeral stem includes a tapered surface tapered toward the cavity of the humeral stem.

In Example 22, the subject matter of Example 21 optionally includes wherein the sensor housing comprises protrusions extending radially outward from the sensor housing, the protrusions located on the sensor housing such that they contact the distal portion of the humeral stem when the sensor housing is installed within the cavity of the humeral stem.

In Example 23, the subject matter of Example 22 optionally includes wherein the protrusions are configured to engage with a tool to install or remove the sensor assembly from the cavity of the humeral stem.

In Example 24, the subject matter of any one or more of Examples 20-23 optionally include wherein the sensor includes an antenna installed within the nose cone.

In Example 25, the subject matter of Example 24 optionally includes wherein the nose cone of the implant plug and the nose cone of the sensor assembly include a mechanical interface configured to help rotate the implant plug and the sensor assembly about their central axes to facilitate coupling or decoupling of the nose cone of the implant plug and the nose cone of the sensor assembly from the humeral stem.

In Example 26, the subject matter of any one or more of Examples 18-25 optionally include an implant plug assembly comprising: an expander plug including: a hollow head defining a portion of a channel within the implant plug assembly; and expansion walls extending from the hollow head and including a distal portion; an expander wedge defining a portion of the channel within the implant plug assembly, the expander wedge including a threaded internal surface; and an expander screw including: a head; and a stem extending from the head, the stem extending through the channel within the implant plug assembly and including a threaded surface.

In Example 27, the subject matter of Example 26 optionally includes wherein the threaded internal surface of the expander wedge and the threaded surface on the stem of the expander screw are complementary such that the threaded surfaces will pull the expander wedge toward the head of the expander screw and the expander wedge will engage with the distal portion of the expansion walls to move the expansion walls radially outward, on condition that the expander screw is rotated clockwise.

In Example 28, the subject matter of any one or more of Examples 26-27 optionally include wherein the threaded internal surface of the expander wedge and the threaded surface on the stem of the expander screw are complementary such that the threaded surfaces will push the expander wedge away from the head of the expander screw and the expander wedge will disengage with the distal portion of the expansion walls to move the expansion walls radially inward, on condition that the expander screw is rotated counterclockwise.

In Example 29, the subject matter of any one or more of Examples 18-28 optionally include a sensor assembly, wherein the sensor assembly comprises: a sensor housing having a coupling interface; and a sensor installed at least partially within the sensor housing, wherein: the cavity in the humeral stem includes a coupling receptacle complementary to the coupling interface of the sensor housing so that the sensor assembly can be removably attached to the humeral stem; and the sensor housing forms an end of the implant when the sensor housing is coupled to the humeral stem.

In Example 30, the subject matter of Example 29 optionally includes wherein the humeral stem comprises an alignment marking indicative of a location of the coupling receptacle.

Example 31 is an apparatus comprising means to implement of any of Examples 1-30.

Example 32 is a system to implement of any of Examples 1-30.

Example 33 is a method to implement of any of Examples 1-30.

In Example 34, the apparatuses or method of any one or any combination of Examples 1-30 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of“ ” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A system configured to be implanted into a human body, the system comprising:
a plurality of stems, each of the plurality of stems having different sizes from one another and are configured for insertion into a bone of a patient, each of the plurality of stems having an elongated body containing a cavity having an opening at an end thereof; and
a sensor assembly configured to removably couple to one or more of the plurality of stems, the sensor assembly including:
a sensor configured for insertion within the cavity; and
a nose cone extending from the sensor and projecting from the cavity to cover the opening when the sensor assembly is coupled with one of the plurality of stems.

2. The system of claim 1, wherein each of the plurality of stems comprises an attachment mechanism formed in the elongated body opposite the cavity, and wherein the attachment mechanism is configured to receive a second implant component wherein the cavity of each of the plurality of stems further comprises an internal surface having threads formed thereon adjacent to the opening of the cavity of each of the plurality of stems, and wherein the sensor assembly includes:
a sensor housing having the sensor therein, the sensor housing is configured to be inserted within the cavity of the elongated body of one or more of the plurality of stems.

3. (canceled)

4. (canceled)

5. The system of claim 2, wherein the sensor housing includes an exterior surface having threads, the threads of the exterior surface of the sensor assembly are complementary to the threads formed on the internal surface of the cavity of the plurality of stems so that the sensor assembly is configured to be screwed into the cavity of one or more of the plurality of stems, and wherein the system includes a first sensor assembly of a first size and a second sensor assembly of a second size, and wherein the nose cone of the first sensor assembly has a first diameter and the nose cone of the second sensor assembly has a second diameter.

6. (canceled)

7. The system of claim 5, further comprising:
a first group of the plurality of stems; and
a second group of the plurality of stems, wherein each stem of the first group of the plurality of stems has a smaller external diameter than each stem of the second group of the plurality of stems,
wherein at least the first sensor assembly is configured to couple with the first group of the plurality of stems and at least the second sensor assembly is configured to couple with the second group of the plurality of stems.

8. The system of claim 7, further comprising:
an implant plug including:
a nose cone; and
a rod extending from the nose cone, the rod has threads formed on an outer surface that are complementary to the threads formed on the internal surface of the cavity of each of the plurality of stems, wherein the implant plug forms an end of each of the plurality of stems when installed in each of the plurality of stems, and wherein the implant plug is configured to provide structural support during assembly of the second implant component and each of the plurality of stems when installed in each of the plurality of stems.

9. The system of claim 8, further including:
a sizing fixture having a plurality of holes, each hole of the plurality of holes corresponds to at least one of external diameters of the elongated body of at least one of the plurality of stems,
wherein each hole of the plurality of holes in the sizing fixture is configured to receive the elongated body of at least one of the plurality of stems and hold the at least one of the plurality of stems while the second implant component is impacted into the attachment mechanism.

10. The system of claim 1, further comprising:
an implant plug assembly including:
an expander plug having:
a hollow head defining a portion of a channel within the implant plug assembly; and
expansion walls extending from the hollow head and including a distal portion;
an expander wedge defining a portion of the channel within the implant plug assembly, the expander wedge having a threaded internal surface; and
an expander screw having:
a head; and
a stem extending from the head, the stem extending through the channel within the implant plug assembly, and including a threaded surface.

11. The system of claim 10, wherein the threaded internal surface of the expander wedge and the threaded surface on the stem of the expander screw are complementary such that the threaded surfaces will pull the expander wedge toward the head of the expander screw and the expander wedge will engage with the distal portion of the expansion walls to move the expansion walls radially outward, on condition that the expander screw is rotated clockwise.

12. The system of claim 10, wherein the threaded internal surface of the expander wedge and the threaded surface on the stem of the expander screw are complementary such that the threaded surfaces will push the expander wedge away from the head of the expander screw and the expander wedge will disengage with the distal portion of the expansion walls to move the expansion walls radially inward, on condition that the expander screw is rotated counterclockwise.

13. The system of claim 1, wherein the sensor assembly includes:
a sensor housing having a coupling interface; and
a sensor installed at least partially within the sensor housing,
wherein:
the cavity of each of the plurality of stems includes a coupling receptacle complementary to the coupling interface of the sensor housing so that the sensor assembly can be removably attached to each of the plurality of stems;
the sensor housing forms an end of the system when the sensor housing is coupled to any of the plurality of stems; and
each of the plurality of stems comprises an alignment marking indicative of a location of the coupling receptacle.

14. (canceled)

15. (canceled)

16. The system of claim 2, further comprising:
an implant plug including:
a nose cone; and
a rod extending from the nose cone, the rod having threads formed on an outer surface that are complementary to the threads formed on the internal surface of the cavity of each of the plurality of stems, wherein the implant plug forms an end of each of the plurality of stems when installed in each of the plurality of stems, and wherein the implant plug is configured to provide structural support during assembly of the second implant component and each of the plurality of stems when the second implant component is installed into each of the plurality of stems;
wherein the nose cone of the implant plug and the nose cone of the sensor assembly each include a mechanical interface configured to help rotate the implant plug and the sensor assembly about their central axes to install or remove the implant plug or the sensor assembly within any one of the plurality of stems.

17. (canceled)

18. An implant that is insertable into a shoulder of a patient, the implant comprising:
a joint interface; and
a humeral stem coupled to the joint interface, the humeral stem including:
an elongated body with a distal portion;
a cavity extending through the distal portion and into the elongated body; and
an attachment mechanism on an opposing end of the humeral stem from the cavity, wherein the attachment mechanism is configured to receive the joint interface.

19. The implant of claim 18, further comprising:
an implant plug having:
a cylindrical body extending between a first end and a second end;
a nose cone extending from the first end of the cylindrical body; and
a shaft extending from the second end of the cylindrical body,
wherein:
the cavity in the humeral stem and the shaft of the implant plug have complementary threads so that the implant plug can be removably attached to the humeral stem; and
the nose cone forms an end of the implant when the implant plug is coupled to the humeral stem.

20. The implant of claim 19, further comprising a sensor assembly, wherein the sensor assembly comprises:
a sensor housing;
a nose cone extending from the sensor housing; and
a sensor installed at least partially within the sensor housing,
wherein:
the cavity in the humeral stem and the sensor housing have complementary threads so that the sensor assembly can be removably attached to the humeral stem; and
the nose cone of the sensor assembly forms an end of the implant when the sensor housing is coupled to the humeral stem.

21. The implant of claim 20, wherein at least a portion of the sensor housing and the nose cone extend outside the humeral stem, and wherein the distal portion of the humeral stem includes a tapered surface tapered toward the cavity of the humeral stem, wherein the sensor housing comprises protrusions extending radially outward from the sensor housing, the protrusions located on the sensor housing such that they contact the distal portion of the humeral stem when the sensor housing is installed within the cavity of the humeral stem, and wherein the protrusions are configured to engage with a tool to install or remove the sensor assembly from the cavity of the humeral stem.

22. (canceled)

23. (canceled)

24. The implant of claim 20, wherein the sensor includes an antenna installed within the nose cone, and wherein the nose cone of the implant plug and the nose cone of the sensor assembly include a mechanical interface configured to help rotate the implant plug and the sensor assembly about their central axes to facilitate coupling or decoupling of the nose cone of the implant plug and the nose cone of the sensor assembly from the humeral stem.

25. (canceled)

26. The implant of claim 18 further comprising:
an implant plug assembly comprising:
an expander plug including:
a hollow head defining a portion of a channel within the implant plug assembly; and
expansion walls extending from the hollow head and including a distal portion;
an expander wedge defining a portion of the channel within the implant plug assembly, the expander wedge including a threaded internal surface; and
an expander screw including:
a head; and
a stem extending from the head, the stem extending through the channel within the implant plug assembly and including a threaded surface.

27. The implant of claim 26, wherein the threaded internal surface of the expander wedge and the threaded surface on the stem of the expander screw are complementary such that the threaded surfaces will pull the expander wedge toward the head of the expander screw and the expander wedge will engage with the distal portion of the expansion walls to move the expansion walls radially outward, on condition that the expander screw is rotated clockwise.

28. The implant of claim 26, wherein the threaded internal surface of the expander wedge and the threaded surface on the stem of the expander screw are complementary such that the threaded surfaces will push the expander wedge away from the head of the expander screw and the expander wedge will disengage with the distal portion of the expansion walls to move the expansion walls radially inward, on condition that the expander screw is rotated counterclockwise.

29. The implant of claim 18, further comprising a sensor assembly,
wherein the sensor assembly comprises:
a sensor housing having a coupling interface; and
a sensor installed at least partially within the sensor housing,
wherein:
the cavity in the humeral stem includes a coupling receptacle complementary to the coupling interface of the sensor housing so that the sensor assembly can be removably attached to the humeral stem;
the sensor housing forms an end of the implant when the sensor housing is coupled to the humeral stem; and
the humeral stem comprises an alignment marking indicative of a location of the coupling receptacle.

30. (canceled)
